(19) United States
(12) Patent Application Publication
(10) Pub. No.: US 2007/0292500 A1
(43) Pub. Date: Dec. 20, 2007

(54) Novel Extended Release Composition of Venlafaxine Hydrochloride Containing Polyvinyl Acetate

(75) Inventors: Sanjay Wagh, Pune (IN); Hidaytulla Aga, Pune (IN); Himadri Sen, Pune (IN)

Correspondence Address: MERCHANT & GOULD PC, P.O. BOX 2903, MINNEAPOLIS, MN, 55402-0903, US

(73) Assignee: LUPIN LTD., (IN)

(21) Appl. No.: 11/597,460

(22) PCT Filed: May 21, 2004
(86) PCT No.: PCT/IN04/00140

§ 371 Date: Jun. 19, 2007

(51) Int. Cl. A61K 9/62; A61K 31/133; A61K 9/64; A61P 25/24; A61K 9/48

(52) U.S. Cl. 424/452; 424/457; 514/653

(57) ABSTRACT

Extended release pharmaceutical composition of Venlafaxine hydrochloride comprising a pharmaceutically acceptable capsule containing minitablets. The minitablets comprise from about 20% to about 70% by weight effective amount of Venlafaxine hydrochloride, polyvinyl acetate, one or more pharmaceutically acceptable excipients. The minitablets have diameter from about 1 mm to 5 mm and are coated with a release controlling composition.

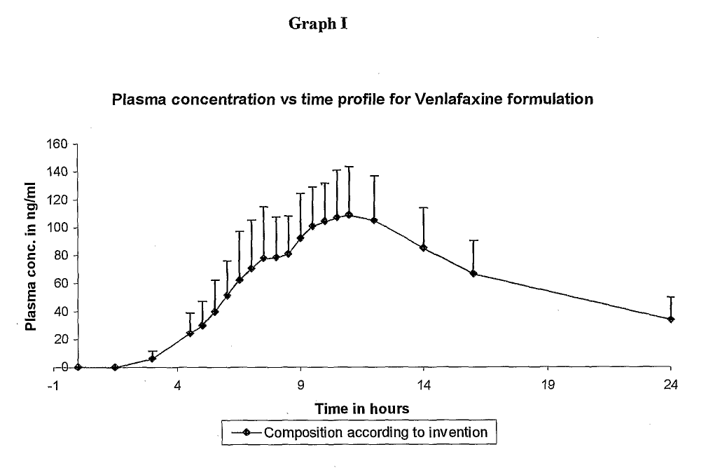

### FIELD OF INVENTION

This invention relates to novel extended release compositions for anti-depressant drug Venlafaxine hydrochloride.

### BACKGROUND OF INVENTION

Venlafaxine hydrochloride or hydrochloride salt of 1-[2-(dimethylamino)-1-(4-methoxyphenyl)ethyl] cyclohexanol is an important drug in the neuropharmacological arsenal used in the treatment of depression.

U.S. Pat. No. 4,535,186, teaches a class of hydroxycycloalkallephenethyl amines as being useful antidepressants and exemplifies Venlafaxine hydrochloride. It is currently marketed in United States as an immediate release tablet as well as an extended release capsule dosage form, under the brand name EFFEXOR® and EFFEXOR XR®.

U.S. Pat. No. 6,274,171 discloses encapsulated formulations of Venlafaxine hydrochloride designed to deliver the drug over an extended period of time. The encapsulated dosage form comprises spheroids of Venlafaxine hydrochloride, microcrystalline cellulose, and optionally hydroxypropyl methylcellulose. These spheroids are coated with a mixture of ethyl cellulose and hydroxypropyl methylcellulose. U.S. Pat. Nos. 6,403,120 and 6,419,958 also disclose similar compositions of Venlafaxine hydrochloride.

PCT application WO 99/22724 also discloses extended release spheroidal compositions. These formulations differ from those in U.S. Pat. No. 6,274,171 in that the spheroids are essentially free from hydroxypropyl methylcellulose.

PCT application WO 03/082262 and related US patent application 2003/0190352 describes extended release dosage form using Venlafaxine base instead of the hydrochloride salt.

PCT applications WO 94/27589 and WO 01/37815 describe osmotic dosage forms containing Venlafaxine hydrochloride.

The formulations discussed above are complicated and require dedicated equipment for manufacturing. Therefore, there exists a need for a cost effective, simple and robust formulation of extended release Venlafaxine.

### OBJECT OF THE INVENTION

It is an object of the present invention to prepare an extended release pharmaceutical composition of Venlafaxine hydrochloride in the form of pharmaceutically acceptable capsules containing minitablets comprised of therapeutically effective amount of Venlafaxine hydrochloride so as to provide a peak blood plasma level of Venlafaxine in more than eight hours, said formulation containing Venlafaxine hydrochloride as the active ingredient and method of preparing such composition.

Another object of the present invention is to provide a method for providing a therapeutic blood plasma concentration of Venlafaxine over a twenty four hour period.

### SUMMARY OF INVENTION

Thus the present invention relates to an extended release pharmaceutical composition of Venlafaxine hydrochloride comprising a pharmaceutically acceptable capsule containing minitablets comprised of therapeutically effective amount of Venlafaxine hydrochloride, an extended release polymer which is polyvinyl acetate and one or more pharmaceutically acceptable excipients, wherein the minitablets are further coated with a release controlling composition comprising polyvinyl acetate, hydrophilic polymer, one or more coating aids.

The present invention further relates to a method for providing a therapeutic blood plasma concentration of Venlafaxine over a twenty four hour period which comprises administering orally to a patient in need thereof, an encapsulated, extended release formulation that provides a peak blood plasma level of Venlafaxine in more than eight hours, said formulation containing Venlafaxine hydrochloride as the active ingredient.

### DETAILED DESCRIPTION OF THE INVENTION

The present invention provides for an extended release pharmaceutical composition comprising Venlafaxine hydrochloride and a process for the preparation thereof, which provides a therapeutic blood plasma level, required for once a day administration. The extended release pharmaceutical composition of the present invention comprises of pharmaceutically acceptable capsules comprising of minitablets containing therapeutically effective amount of Venlafaxine hydrochloride, an extended release polymer which is polyvinyl acetate and optionally one or more pharmaceutically acceptable excipients. The minitablets of the present invention may further be coated with a composition comprising polyvinyl acetate, a hydrophilic polymer, a plasticizer and other coating aids such as fillers, anti-sticking agents, glidants and colorants.

The formulation contains from about 10 to about 400 mg of Venlafaxine. Venlafaxine may be present either in the form of freebase or its pharmaceutically acceptable salt.

Venlafaxine hydrochloride used according to the present invention is present in the range of about 20% to about 70% by weight of the formulation. Polyvinyl acetate is present in the formulation in the range of about 2% to about 20% by weight. Polyvinyl acetate is available commercially in dry powder form or as 30% aqueous dispersion under the brand Kollicoat SR 30D marketed by BASF.

The core may also contain other pharmaceutically acceptable excipients in the range of 20% to 60%. The excipient may include fillers, binders, swelling excipients, glidants, lubricants etc.

Pharmaceutical excipients used in the present invention include fillers which may be selected from lactose, starch, saccharose, glucose, microcrystalline cellulose, mannitol, sorbitol, calcium hydrogen phosphate, aluminium silicate, sodium chloride and combinations thereof; binders which may be selected from starch, gelatin, carboxymethylcellulose, polyvinylpyrrolidine, crosslinked polyvinylpyrrolidone, sodium alginate, microcrystalline cellulose or combinations thereof; glidants which may be selected from magnesium sterate, calcium stearate, aluminum stearate, stearic acid, palmitic acid, sterol, polyethylene glycol, talc, etc.; one or more lubricants which may be selected from stearic acid, calcium stearate, magnesium stearate or aluminum stearate, talc, colloidal silicon dioxide, etc.

The film coating comprises a combination of polyvinyl acetate and a hydrophilic polymer along with one or more plasticizers and other coating aids such as fillers, anti-sticking agents, antifoaming agents, colorants etc.

The coating is preferably present in the composition in the range of 1% to 25% by weight, with respect to the weight of the core tablet.

The hydrophilic polymer used in the coating of the present invention may be selected from the group consisting of polyvinyl pyrrolidone, hydroxypropyl methylcellulose, hydroxypropyl cellulose, hydroxyethyl cellulose, methylcellulose, and the like.

Plasticizers used in the coating of the present invention may be selected from acetyl tributyl citrate, acetyl triethyl citrate, acetylated fatty acid glycerides, castor oil, diethyl phthalate, diethyl sebacate, dibutyl sebacate, dimethyl phthalate, glycerol, glyceryl monostearate, glyceryl triacetate, polyoxyethylene/polyoxypropylene copolymers, polyethylene glycol, triethyl citrate, dibutyl phthalate, oils, and propylene glycol.

Fillers used in the coating of the present invention may be selected from lactose, polydextrose and maltodextrin; one or more anti-sticking agents used in the present invention may be selected from talc, magnesium stearate, calcium stearate, colloidal silicon dioxide etc.; one or more colorants such as titanium dioxide, iron oxide, lakes etc.

According to the invention, an active ingredient and filler are blended together and granulated with an aqueous dispersion of polyvinyl acetate. The obtained granules are dried preferably at a temperature between 40° C. and 70° C. The dried granules are sized and blended with lubricants and/or glidants and compressed into minitablets. The minitablets have a diameter of 1-5 mm. Optionally the active ingredient, filler and polyvinyl acetate may be dry granulated and compressed into minitablets, or directly compressed into minitablets.

In the second step of the process, these minitablets are coated with polyvinyl acetate and hydrophilic polymer, along with one or more plasticizers and other coating aids such as fillers, anti-sticking agents, antifoaming agents, colorants etc. The coating may be performed using dispersion or colloidal solution. Coating dispersion can be prepared either by mixing powders of polymers or other suitable ingredients in organic solvents or in combinations of organic solvents or by mixing and diluting aqueous dispersions of polymers with water. In the next step, plasticizer or a combination of plasticizers may optionally be added, followed by antisticking agents, colorants and fillers. The coating can be performed by means known to those skilled in the art.

The coated tablets may optionally be subjected to the process of curing. This was achieved by heating the tablets at about 35° C. to about 50° C. for about 6 to about 12 hours.

These coated minitablets are then filled into pharmaceutically acceptable hard gelatin capsules using techniques well known to those skilled in the art. One or more minitablets are loaded into a single hard gelatin capsule to provide a unit dose. Most preferably the minitablets provide additive amounts of Venlafaxine without modifying the release profile. For example, by making a round tablet containing 18.75 mg of Venlafaxine, capsules containing 37.5 mg, 75 mg and 150 mg of Venlafaxine can be formed by standard filling of capsule with 2, 4 or 8 minitablets.

The invention will be further described with reference to the following non-limiting Examples:

TABLE 1

| Name of ingredient | Example 1 | Example 2 | Example 3 | Example 4 |
|---|---|---|---|---|
| Core Formula | % w/w | % w/w | % w/w | % w/w |
| Venlafaxine HCl eq. To | 47.6 | 47.60 | 47.6 | 47.6 |
| Venlafaxine 18.75 mg | | | | |
| Microcrystalline cellulose | 42.9 | 49.90 | 40.9 | 42.9 |
| Polyvinyl acetate | 8.0 | — | 10.0 | 8.0 |
| (Kollicoat SR 30 D) | | | | |
| Magnesium Stearate | 1.5 | 2.50 | 1.5 | 1.5 |
| Total | 100 | 100 | 100 | 100 |
| Coating | % solid content | % solid content | % solid content | % solid content |
| Polyvinyl acetate | 66.7 | 66.66 | 57.69 | 52.63 |
| (Kollicoat SR 30 D) | | | | |
| Povidone (PVP K 17) | 17.8 | 20.0 | 30.77 | 35.09 |
| Colloidal silicon dioxide | 4.4 | 2.22 | 1.92 | 3.51 |
| (Aerosil) | | | | |
| Propylene glycol | 11.1 | 11.11 | 9.62 | 8.77 |
| Total | 100 | 100 | 100 | 100 |
| % Coating build up | 9-11% | 6.0% | 10% | 15% |
| Curing | 45° C. for 12 hrs | 45° C. for 12 hrs | 45° C. for 12 hrs | 45° C. for 12 hrs |

TABLE 2

| Name of ingredient | Example 5 | Example 6 | Example 7 | Example 8 |
|---|---|---|---|---|
| Core Formula | % w/w | % w/w | % w/w | % w/w |
| Venlafaxine HCl eq. To | 47.6 | 47.6 | 30.6 | 53.45 |
| Venlafaxine 18.75 mg | | | | |
| Calcium hydrogen | 42.9 | 42.9 | 57.19 | — |
| phosphate/MCC | | | | |
| Starch | — | — | — | 42.87 |
| Polyvinyl acetate | 8.0 | 8.0 | 10.71 | 2.0 |
| (Kollicoat SR 30 D) | | | | |
| Magnesium Stearate | 1.5 | 1.5 | 1.5 | 1.68 |
| Total | 100 | 100 | 100 | 100 |
| Coating | % solid content | % solid content | % solid content | % solid content |
| Polyvinyl acetate | 57.69 | 66.7 | 66.7 | 66.66 |
| (Kollicoat SR 30 D) | | | | |
| Povidone (PVP K 17) | 30.77 | 17.8 | 17.8 | 20.00 |
| Colloidal silicon dioxide | 3.57 | 4.4 | 4.4 | 2.22 |
| Propylene glycol | 9.62 | 11.1 | 11.1 | 11.11 |
| Total | 100 | 100 | 100 | 100 |
| % Coating build up | 10% | 9-11% | 10% | 20% |
| Curing | 45° C. for 12 hrs | No curing | 45° C. for 12 hrs | 45° C. for 12 hrs |

Process: Granulate Venlafaxine hydrochloride and Microcrystalline cellulose/calcium hydrogen phosphate/starch with polyvinyl acetate. Dry the granules, lubricate and compress into minitablets. Coat the minitablets with coating solution b) to a weight gain of about 4% to about 15%. Cure the tablets at 45° C. for 12 hours. Fill 2 minitablets in size ‘3’ capsule shells for 37.5 mg product strength. Alternately fill 4 minitablets in size ‘2’ capsule shells for 75 mg product strength or 8 minitablets in size ‘0’ capsule shells for 150 mg product strength.

The formulations exemplified above were studied for dissolution release. Dissolution studies were performed using USP 1 apparatus, 100 rpm and using 900 ml of purified water. Results of the dissolution profile are summarized in table 3:

TABLE 3

| Time | Examples | | | | | | | | |
|---|---|---|---|---|---|---|---|---|---|
| (hours) | EffexorXR ® | 1 | 2 | 3 | 4 | 5 | 6 | 7 | 8 |
| 2 | 20.9 | 11.9 | 11.9 | 5.7 | 25.6 | 6.3 | 34.8 | 27.6 | 11.0 |
| 4 | 53.0 | 29.5 | 40.8 | 24.7 | 57.4 | 24.0 | 69.6 | 54.7 | 32.0 |
| 6 | 72.4 | 50.9 | 60.4 | 43.7 | 76.5 | 41.9 | 83.6 | 72.7 | 57.3 |
| 8 | 84.3 | 69.3 | 72.7 | 62.1 | 92.2 | 60.3 | 92.0 | 89.7 | 72.9 |
| 12 | 97.6 | 88.6 | 88.5 | 86.0 | 100.0 | 84.4 | 98.3 | 100.2 | 94.9 |
| 16 | 104.5 | 96.9 | 97.3 | 99.7 | 102.6 | 96.8 | 99.3 | — | — |
| 24 | 111.5 | 102.5 | 102.2 | 106.1 | 105.3 | 108.9 | — | 102.5 | 99.6 |

The formulation as per example 1 of the present invention was subjected to bioavailability study and was evaluated for the commonly studied pharmacokinetic parameters such as Area under the plasma concentration vs time profile (AUC), peak plasma concentration (Cmax) and time to reach peak plasma concentration (Tmax). The profile exhibits characteristics required for once a day formulations with Tmax of about 11 hours as shown in graph 1 of the accompanying drawing.

1. An extended release pharmaceutical composition of Venlafaxine hydrochloride comprising a pharmaceutically acceptable capsule containing minitablets, each said minitablets comprising therapeutically effective amount of Venlafaxine hydrochloride, polyvinyl acetate, one or more pharmaceutically acceptable excipients, said minitablets being further coated with a release controlling composition.

2. An extended release pharmaceutical composition as claimed in claim 1 wherein the minitablets have diameter from about 1 mm to 5 mm.

3. An extended release pharmaceutical composition according to claim 1, wherein Venlafaxine hydrochloride is present in the range of about 20% to about 70% by weight of minitablets.

4. An extended release pharmaceutical composition according to claim 1, wherein polyvinyl acetate in the minitablets is present in the range of about 2% to about 20% by weight of minitablets.

5. An extended release pharmaceutical composition of Venlafaxine hydrochloride according to claim 1, wherein the pharmaceutically acceptable excipients may be selected from fillers, binders, glidants, and lubricants.

6. An extended release pharmaceutical composition of Venlafaxine hydrochloride according to claim 5, wherein the fillers are selected from lactose, starch, saccharose, glucose, microcrystalline cellulose, mannitol, sorbitol, calcium hydrogen phosphate, aluminium silicate, sodium chloride and combinations thereof.

7. An extended release pharmaceutical composition of Venlafaxine hydrochloride according to claim 5, wherein the binders are selected from starch, gelatin, carboxymethylcellulose, polyvinylpyrrolidine, crosslinked polyvinylpyrrolidone, sodium alginate, microcrystalline cellulose or combinations thereof.

8. An extended release pharmaceutical composition of Venlafaxine hydrochloride according to claim 5, wherein the glidants are selected from magnesium stearate, calcium stearate, aluminum stearate, stearic acid, palmitic acid, sterol, polyethylene glycol, talc and combinations thereof.

9. An extended release pharmaceutical composition of Venlafaxine hydrochloride according to claim 5, wherein the lubricants which are from stearic acid, calcium stearate, magnesium stearate, aluminum stearate, talc, colloidal silicon dioxide and combinations thereof.

10. An extended release pharmaceutical composition according to claim 1, wherein the total weight of the release controlling coating composition is in the range of about 5% to about 25% by weight.

11. An extended release pharmaceutical composition as claimed in claim 1, wherein the release controlling coating composition comprises polyvinyl acetate, hydrophilic polymer, one or more plasticizers and one or more coating aids.

12. An extended release pharmaceutical composition according to claim 11, wherein the release controlling coating composition comprises from about 25% to about 75% of polyvinyl acetate by weight.

13. An extended release pharmaceutical composition according to claim 11, wherein the release controlling coating composition comprises from about 10% to about 40% of hydrophilic polymer by weight.

14. An extended release pharmaceutical composition according to claim 11, wherein the hydrophilic polymer in the coating is selected from the group consisting of polyvinyl pyrrolidone, hydroxypropyl methylcellulose, hydroxypropyl cellulose, hydroxyethyl cellulose, methyl cellulose, and the like.

15. An extended release pharmaceutical composition according to claim 11, wherein the plasticizer is selected from the group consisting of polyethylene glycol, glyceryl triacetate, triethyl citrate, dibutyl phthalate, oils, propylene glycol, and the like.

16. An extended release pharmaceutical composition according to claim 11, wherein the coating aids are selected from the group consisting of fillers, antisticking agents, glidants and colorants.

17. An extended release pharmaceutical composition according to claim 1, wherein the capsules are hard gelatin capsules.

18. An extended release pharmaceutical composition according to claim 17, wherein each hard gelatin capsule contains one to ten minitablets.

19. A process for the preparation of an extended release pharmaceutical composition of Venlafaxine, comprising granulation of Venlafaxine hydrochloride and pharmaceutically acceptable excipients with polyvinyl acetate, compressing into minitablets, coating the minitablets with a dispersion of polyvinyl acetate, hydrophilic polymer, one or more coating aids and further encapsulating into pharmaceutically acceptable hard gelatin capsules.

20. A process for the preparation of an extended release pharmaceutical composition according to claim 19, wherein from one to ten minitablets are encapsulated.

21. A process for the preparation of an extended release pharmaceutical composition according to claim 19, wherein the minitablets are prepared by wet granulation, dry granulation or by direct compression.

22. A method for providing a therapeutic blood plasma concentration of Venlafaxine over a twenty four hour period which comprises administering orally to a patient in need thereof, an encapsulated, extended release formulation that provides a peak blood plasma level of Venlafaxine in more than eight hours, said formulation containing Venlafaxine hydrochloride as the active ingredient.

23. A method for providing a therapeutic blood plasma concentration of Venlafaxine over a twenty four hour period which comprises administering orally to a patient in need thereof, an encapsulated, extended release formulation that provides a peak blood plasma level of Venlafaxine in about 8-12 hours, said formulation containing Venlafaxine hydrochloride as the active ingredient.

24. An extended release pharmaceutical composition of Venlafaxine hydrochloride for providing a therapeutic blood plasma concentration of Venlafaxine over a twenty four hour period which comprises administering orally to a patient in need thereof, an encapsulated, extended release formulation that provides a peak blood plasma level of Venlafaxine in more than eight hours, said formulation containing Venlafaxine hydrochloride as the active ingredient.

25. An extended release pharmaceutical composition of Venlafaxine hydrochloride for providing a therapeutic blood plasma concentration of Venlafaxine over a twenty four hour period which comprises administering orally to a patient in need thereof, an encapsulated, extended release formulation that provides a peak blood plasma levels of Venlafaxine in about 8-12 hours, said formulation containing Venlafaxine hydrochloride as the active ingredient.
